(19) United States
(12) Patent Application Publication
(10) Pub. No.: US 2007/0179447 A1
(43) Pub. Date: Aug. 2, 2007

(54) Drooped hook device for placing a cannula in a vein

(75) Inventors: Jean-Luc Carrez, Ecouen (FR); Valery Dalle, Gouvieux (FR); Pierrick Guyomarc'h, Ermont (FR); Jean-Max Huet, Clichy (FR)

Correspondence Address: LEVINE & MANDELBAUM, 444 MADISON AVENUE, 35TH FLOOR, NEW YORK, NY, 10022, US

(73) Assignee: Vygon, Ecouen (FR)

(21) Appl. No.: 10/598,429

(22) PCT Filed: Feb. 2, 2005
(86) PCT No.: PCT/FR05/00221

§ 371 Date: Oct. 16, 2006

(30) Foreign Application Priority Data
Mar. 2, 2004 (FR) 0402124

(51) Int. Cl. A61M 5/178

(52) U.S. Cl. 604/164.09

(57) ABSTRACT

The invention relates to placing a cannula by means of a needle and a non-piercing cage. A hook is formed on the end (6a) of a holding member (6) which is pivotally mounted on a non piercing cage (5) around a transversal axis pivoting in the needle sliding direction in order to be engaged with the flange (12) of the cannula base. An opposite end (6b) of said member laterally contacts the needle when said needle penetrates in the cage. The member is embodied in such a way that it is pivotable about said axis when it is not in contact with the needle in such a way the hook is raised and disengaged from the flange of the base while the opposite end of the member is lowered and interposed in front of the end of the needle puncture, thereby preventing the end of exiting from a chamber through the distal exit thereof. Said invention can be used for intravenous catheters.

This present invention concerns devices used for the insertion into a vein of a cannula composed of a short tubular catheter with a proximal base, by means of a puncture needle.

The insertion procedure includes a puncture stage in which the needle is pushed into the catheter base and into the catheter so that its point exits at the distal end of the catheter, and in which, with this point, the operator punctures the vein into which he wishes to insert the catheter, with an insertion stage in which the operator slides the catheter along the needle in the distal direction causing the catheter to enter into the vein, and a withdrawal stage in which the operator withdraws the needle from the vein, from the catheter and from the catheter base.

At the end of the withdrawal stage, the point of the needle is exposed to the open air and there is a risk that the operator who is holding the catheter and its base with one hand and who is holding the needle with the other hand, has inadequate control over the needle and can pierce his skin with its point.

In order to remove this risk, a known arrangement temporarily attaches, on the extension toward the rear of the catheter base, a detachable cage through which the needle can slide and which is equipped with a trap to hold the puncture end of the needle in the chamber when the latter exits from the base, and to remain in position on this end when the cage is detached from the base.

In order to attach the cage temporarily onto the base of the needle, a known arrangement creates a friction-type conical interlocking of the cage in or on the base of the needle, so that the cage detaches from the base under the effect of traction applied axially to the needle after the puncture end of the needle has entered into the cage (EP 0 456 694 or U.S. Pat. No. 5,322,517, U.S. Pat. No. 5,135,504, U.S. Pat. No. 5,176,655, and others).

The risk still exists however, if the cage detaches from the base prematurely before the puncture end of the needle has been trapped in the cage.

In order to remove this risk, it has been recommended that the cage be fitted with a hook that is mobile transversally, held by the needle in a retention position in which it is locked to the catheter base, and capable of returning of its own accord to a position of freedom when the puncture end of the needle is withdrawn into the cage.

Publication EP 0 891 198 or U.S. Pat. No. 6,001,080 achieves this retention by penetration, into a cavity formed on the inner face of the wall of the catheter base, by a dog of the cage, the said dog being held in a retention position by a lateral contact with the needle and being released and capable of moving transversally in the cage to escape from the cavity when this lateral contact is eliminated by withdrawal of the needle behind the dog.

This retention arrangement, which is totally concealed in the base and in the cage, is difficult to control, and there is a risk that the automatic radial movement of the dog may be insufficient to free the cage from the base.

U.S. Pat. No. 6,234,999 describes another retention arrangement in which the cage has an external hooking device held by an external collar of the base, but which is not held by the needle, so that an unintended traction on the cage can end this retention prematurely.

U.S. Pat. No. 6,629,959 B2 describes constrained spring systems which are triggered on removal of the needle, and a cage/cannula locking system.

Publication EP 1 350 538 A1 describes a constrained spring system for a needle alone.

U.S. Pat. No. 5,147,327 describes a system for a needle alone with a duct sliding on a metal tube.

U.S. Pat. No. 5,183,468 describes a cage with a lever which tilts on the emergence of the needle, resulting in a gripping action around of the needle. Publication PCT WO 94/00172 describes a system with a flexible duct and pre-constrained spring element.

This present invention has as its objective to provide a cage equipped with a retention arrangement which overcomes the aforementioned drawbacks, and in particular a retention arrangement which is visible both at the outside of the cage and of the base, and held by the needle in a retention position for as long as the puncture end of the needle is not withdrawn into the cage.

One subject of the invention is therefore an arrangement for the insertion into a vein of a cannula composed of a short catheter with a proximal base, where this arrangement includes a needle with a puncture end and anti prick cage which extends the base in the proximal direction, where this chamber forms a chamber through which the needle slides and which is equipped with a trap to hold the puncture end of the needle in the chamber when the needle is withdrawn from the catheter base, with the cage and the base being equipped with retention resources that combine so as to perform temporary retention of the cage and of the base before the puncture end of the needle is trapped in the chamber of the cage, with the said retention resources including an external rim formed on the base and an external dog provided on the cage to be held by this rim, characterised in that this dog is formed at one end of a retention device mounted to tilt in the cage around a pivoting axis which is transverse to the sliding direction of the needle, the said device having one opposite end in lateral contact with the needle when the latter traverses the cage and where the said device is designed to tilt around the said axis when it is no longer in contact with the needle, so that the dog lifts and releases itself from the rim of the base while the said opposite end of the device drops and positions itself in front of the puncture end of the needle, preventing this end from exiting from the chamber via the distal end of the latter.

In particular implementations, the arrangement of the invention has one or more of the following characteristics:
the retention device is designed so that the weight of the part of the retention device located between the pivoting axis and the retention end is less than the weight of the part of the device located between this axis and the said end;
the pivoting axis is composed of lateral nipples formed on the retention device and which are designed to pivot in a cradle created by cut outs in two opposite walls formed on the cage on either side of the retention device;
the cradle accommodates the nipples by a click-on action;
the retention device has a flexible tongue which is held compressed elastically by a wall of the cage when the device is held by the needle and which deploys under this wall when the device has tilted as a result of withdrawing the needle, so that tilting of the device in the reverse direction is prevented by trapping this tongue under the said wall.

The following is a description, by way of an example, of one implementation of an arrangement according to the invention, with reference to figures in the attached drawings, in which:

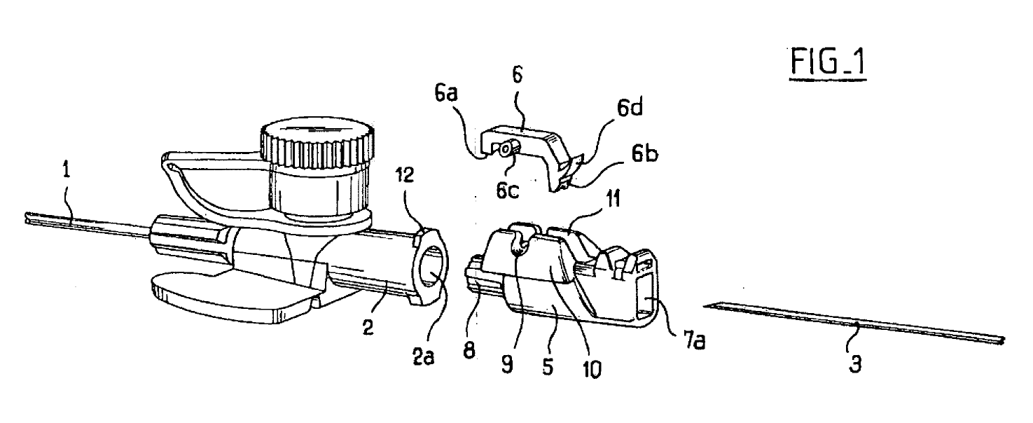

FIG. 1 is a view in external perspective of the arrangement in which the short catheter with its base, the needle, the cage and the retention have been shown separately;

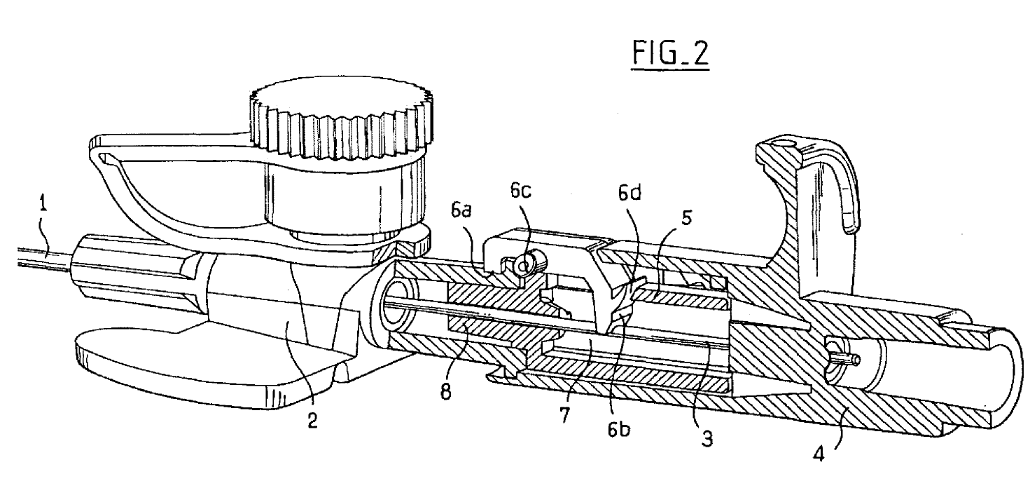

FIG. 2 is a partial view in perspective with a section of the arrangement in which it is also possible to see the base of the needle, the retention device being in a retention position of the cage on the base;

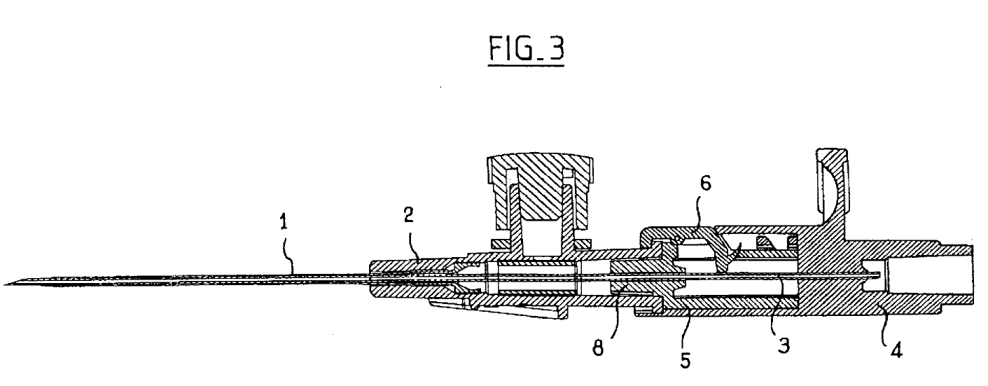

FIG. 3 is a longitudinal section of the arrangement of FIG. 2;

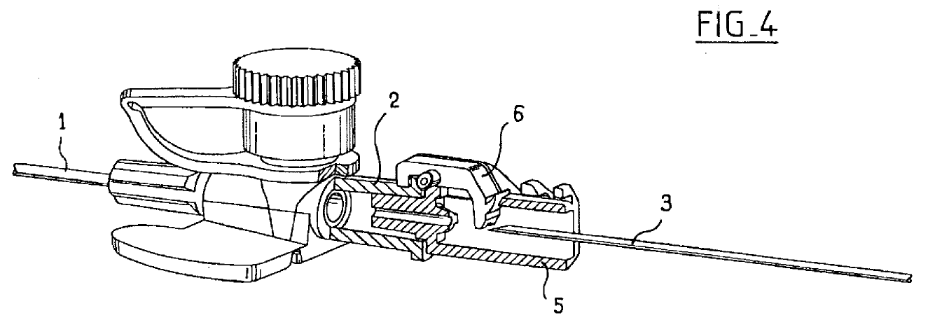

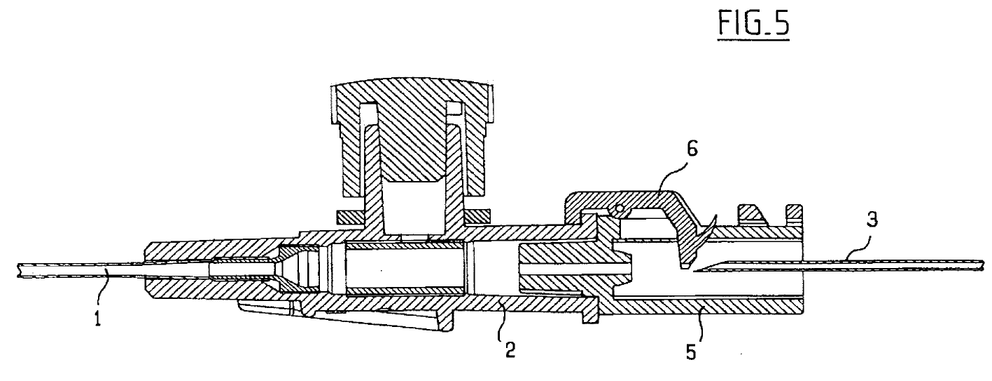

FIGS. 4 and 5 are views in perspective with a partial section of the arrangement when the needle is withdrawn;

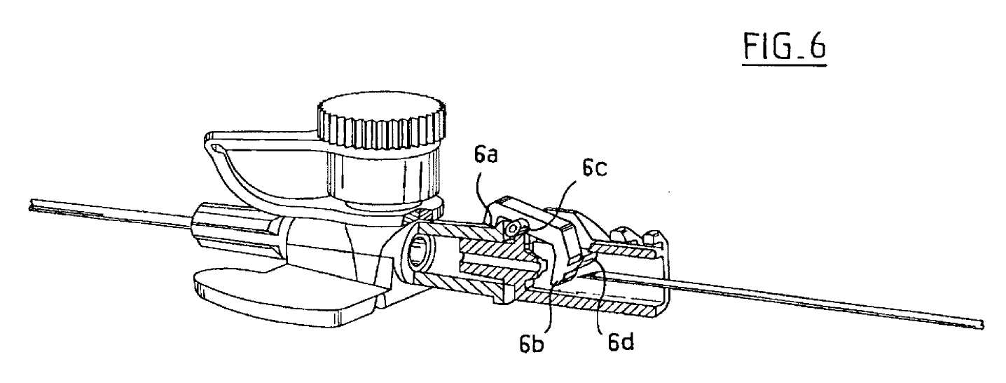

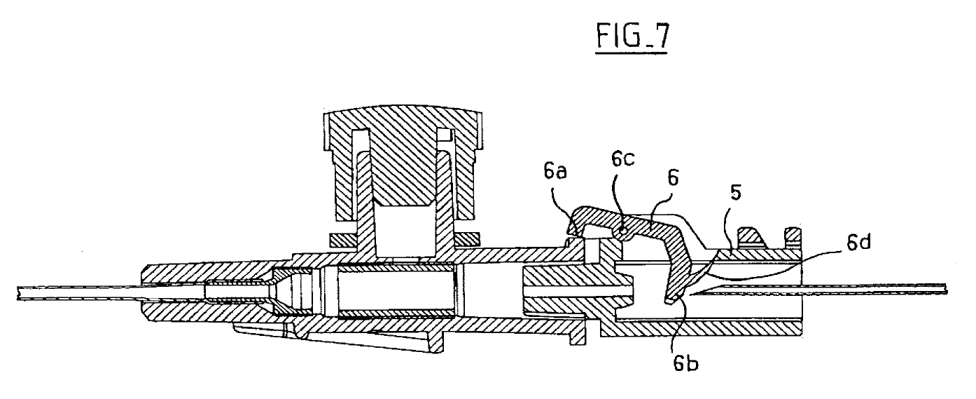

FIGS. 6 and 7 are views in perspective and in section of the arrangement when the retention device has tilted, and

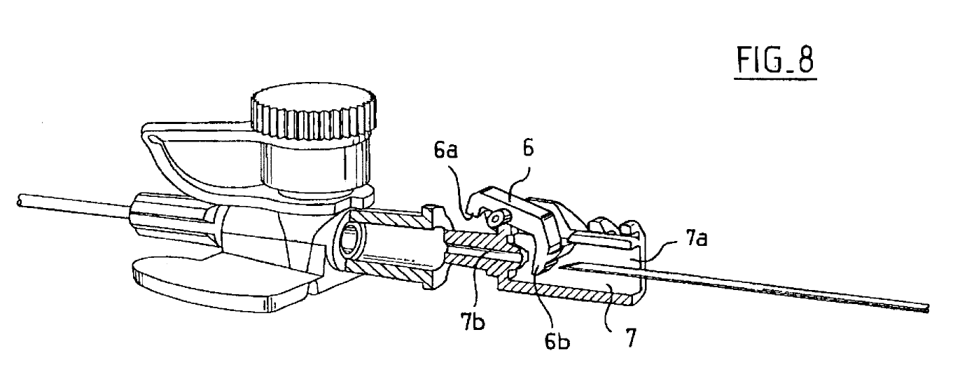

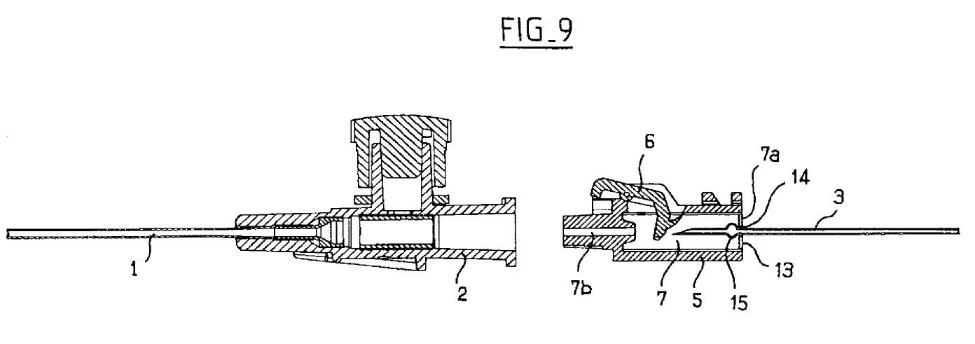

FIGS. 8 and 9 are views in perspective and in section of the arrangement when the cage is detached from the catheter base.

The arrangement shown in the figures includes:
a short catheter (1) equipped with a proximal base (2);
a puncture needle (3) equipped with a proximal base (4);
a protective cage (5) equipped with a retention device (6).

In a manner which is known of itself, the cage forms a chamber (7) for passage of needle, which has a proximal entrance (7a) and a distal exit (7b), and around the distal exit (7b), the cage forms a nose (8) which is able to interlock with or without friction in the entrance (2a) of the base (2) of the short catheter.

The retention device (6), created from synthetic resin for example, has:
a retention end (6a),
an opposite end (6b) for lateral contact with the needle,
two coaxial transverse nipples (6c),
a flexible tongue (6d).

The two nipples form a pivoting axis, and can be accommodated by a click-on action in a cradle (9) formed by cut-outs in two opposite walls (10, 11) of the cage (5) and between which the retention device is able to tilt (6).

Toward its entrance, the catheter base has a continuous or discontinuous external collar (12) with which the retention end of the lever can interlock.

Initially (FIG. 3), the needle traverses the cage, traverses the cannula, and emerges at the end of the catheter tube. In this situation, the cage is held between the catheter base, in which the nose of the cage is accommodated, and the base (4) of the needle, while the retention device (6) is held in a retention position by the needle.

After skin puncture and insertion of the catheter into the vein, the needle is withdrawn and its end arrives in the cage as it emerges from the device (FIGS. 4 and 5).

The device, which is no longer held by the needle, tilts of its own accord (FIGS. 6 and 7), and its flexible tongue (6d) deploys under the lateral wall of the cage (5), preventing reverse tilting of the device.

The end (6b) of the device is then located between the needle and the exit (7b) of the chamber (7) preventing the needle from leaving the cage via this exit (FIGS. 8 and 9). Preferably, resources are also provided in a manner which is known of itself, to prevent the puncture end of the needle from leaving the cage via the proximal entrance of the chamber.

These resources, many examples of which are known, have not been shown in FIGS. 1 to 8 in order to avoid confusion.

It has been proposed that the cage be connected to the base of the needle by a deployable link so that in the deployed state, the length of the link is less than the length of the needle (WO 94/00172, U.S. Pat. No. 5,176,655, U.S. Pat. No. 6,234,999, U.S. Pat. No. 6,001,080).

It has also been proposed that the proximal entrance of the cage be equipped with a transverse wall containing a hole for the passage of the needle, and that the needle be equipped with a local bulge ahead of this hole in the direction of the catheter base so that sliding the needle in the proximal direction is halted by jamming of this bulge against the periphery of the hole.

This present invention is not based on any particular design of such a retention arrangement and, purely as an example, FIG. 9 shows an arrangement composed of a fixed transverse wall (13) equipped with a hole (14) to stop any local movement (15) of the needle.

The invention is not limited to the implementations that have been described.

1. An arrangement for the insertion into a vein of a cannula composed of a short tubular catheter (1) with a proximal base (2), where this arrangement includes a needle (3) which has a skin-puncture end (3a) and anti-prick cage (5) which extends the base in the proximal direction, where this chamber forms a chamber (7) through which the needle slides from a proximal entrance (7a) to a distal exit (7b) of the chamber, and is equipped with a trap to hold the puncture end of the needle in the chamber when the needle is withdrawn from the catheter base, with the cage and the base being equipped with retention resources that combine so as to perform temporary retention of the cage on the catheter base before the puncture end of the needle is trapped in the chamber of the cage, with the said retention resources including an external rim (12) formed on the base and an external dog (6a) provided on the cage to be held by this rim (12), characterised in that this dog is formed at one end (6a) of a retention device (6) mounted to tilt in the cage around a pivoting axis which is transverse to the sliding direction of the needle, the said device having one opposite end (6b) in lateral contact with the needle when the latter traverses the cage and the said device being designed to tilt around the said axis when it is no longer in contact with the needle, so that the dog lifts and releases itself from the rim of the base while the said opposite end of the device drops and positions itself in front of the puncture end of the needle, preventing this end from exiting from the chamber via the distal end (7b) of the chamber.

2. An arrangement according to claim 1, in which the retention device (6) is designed so that the weight of the part of the device located between the pivoting axis and the retention end (6a) is less than the weight of the part of the device located between this axis and the said contact end (6b).

3. An arrangement according to claim 1, in which the said pivoting axis is composed of lateral nipples (6c) formed on the device, and which are accommodated in a cradle (9) created by cut-outs in two opposite walls (10, 11) formed on the cage on either side of the retention device.

4. An arrangement according to claim 3 in which the said cradle (9) accommodates the said nipples (6c) by a clickon action.

5. An arrangement according to claim 1 in which the said retention device (6) has a flexible tongue (6d) which is held compressed elastically by a wall of the cage when the device is held by the needle, and which deploys under this wall when the lever has tilted as a result of withdrawing the needle, so that tilting of the device in the reverse direction is prevented by trapping this tongue under the said wall.

6. An arrangement according to claim 1, and which includes resources (13, 15) to prevent the puncture end of the needle from leaving the cage via the proximal entrance (7a) of the chamber.

7. An arrangement according to claim 2, in which the said pivoting axis is composed of lateral nipples (6c) formed on the device, and which are accommodated in a cradle (9) created by cut-outs in two opposite walls (10, 11) formed on the cage on either side of the retention device.

8. An arrangement according to claim 7 in which the said cradle (9) accommodates the said nipples (6c) by a clickon action.

9. An arrangement according to claim 2 in which the said retention device (6) has a flexible tongue (6d) which is held compressed elastically by a wall of the cage when the device is held by the needle, and which deploys under this wall when the lever has tilted as a result of withdrawing the needle, so that tilting of the device in the reverse direction is prevented by trapping this tongue under the said wall.
